(19) United States
(12) Patent Application Publication
(10) Pub. No.: US 2014/0094679 A1
(43) Pub. Date: Apr. 3, 2014

(54) SYSTEMS AND METHODS FOR PERFORMING ORGAN DETECTION

(71) Applicants: Ferenc Kovacs, Szeged Csongrad (HU); Andras Kriston, Szeged Csongrad (HU); Tamas Blaskovics, Budaors (HU); William J. Bridge, Waukesha, WI (US); Robert John Johnsen, Waukesha, WI (US)

(72) Inventors: Ferenc Kovacs, Szeged Csongrad (HU); Andras Kriston, Szeged Csongrad (HU); Tamas Blaskovics, Budaors (HU); William J. Bridge, Waukesha, WI (US); Robert John Johnsen, Waukesha, WI (US)

(73) Assignee: General Electric Company, Schenectady, NY (US)

(21) Appl. No.: 13/644,073

(22) Filed: Oct. 3, 2012

(51) Int. Cl. G06K 9/34; A61B 6/00

(52) U.S. Cl. 600/407; 382/131

(57) ABSTRACT

A method for automatically detecting an organ of interest that includes accessing a medical image dataset using a processor, automatically segmenting the medical image dataset to identify an outline of a body of a patient, automatically determining an axial reference image slice and a axial center point using the segmented body of the patient, automatically determining a location of the organ of interest using the axial reference image slice and the axial center point, and automatically placing a visual indicator in the organ of interest based on the determined location. A medical imaging system and a non-transitory computer readable medium are also described herein.

### BACKGROUND OF THE INVENTION

The subject matter disclosed herein relates generally to imaging systems, and more particularly, to systems and methods for performing a fully automatic cross-modality detection of an organ of interest.

In an oncology examination, a patient may go through a series of examinations, using for example, a positron emission tomography (PET) system, a single photon emission computed tomography (SPECT) system, a computed tomography (CT) system, an ultrasound system, an x-ray system, a magnetic resonance (MR) system, and/or other imaging systems. The series of examinations is performed to continuously monitor the patient's response to treatment. When evaluating a patient's response to treatment, the previous and follow-up examinations are often analyzed together. The results from the analysis of the follow-up examination may be saved together with results of the analysis of the previous examination(s). Accordingly, information on the progression of the disease throughout the whole series of examinations may be available to the clinician at any time from the same file and/or location.

However, in some cases, the physician may desire to perform the follow-up examination to acquire only functional information. For example, the physician may desire to perform the follow-up examination using a PET system or a SPECT system. However, when analyzing PET images, or other functional images, it may be difficult to identify an object of interest, such as for example, the liver, when comparing the liver for the same patient over time. Currently, to identify the liver in a PET image, the user manually selects the type of the segmentation to be performed on the correspondent anatomical image pair. The user then manually draws a seed region in the liver to perform the segmentation. Optionally, the user may manually draw a region of interest (ROI) inside the liver to perform measurements. However, the manual segmentation methods are user intensive and can increase the time required for the physician to read a diagnosis.

### BRIEF DESCRIPTION OF THE INVENTION

In one embodiment, a method for automatically detecting or displaying an organ of interest is provided. The method includes accessing a medical image dataset using a processor, automatically segmenting the medical image dataset to identify an outline of a body of a patient, automatically determining an axial reference image slice and a axial center point using the segmented body of the patient, automatically determining a location of the organ of interest using the axial reference image slice and the axial center point, and automatically placing a visual indicator in the organ of interest based on the determined location.

In another embodiment, a medical imaging system is provided. The medical imaging system includes a detector array and a computer coupled to the detector array. The computer is configured to access a medical image dataset using a processor, automatically segment the medical image dataset to identify an outline of a body of a patient, automatically determine an axial reference image slice and a axial center point using the segmented body of the patient, automatically determine a location of a liver using the axial reference image slice and the axial center point, and automatically place a visual indicator in the organ of interest based on the determined location.

In a further embodiment, a non-transitory computer readable medium is provided. The non-transitory computer readable medium is programmed to instruct a computer to access a medical image dataset using a processor, automatically segment the medical image dataset to identify an outline of a body of a patient, automatically determine an axial reference image slice and an axial center point using the segmented body of the patient, automatically determine a location of a liver using the axial reference image slice and the axial center point, and automatically placing a visual indicator in the organ of interest based on the determined location.

### BRIEF DESCRIPTION OF THE DRAWINGS

FIG. 1 is a flowchart of an exemplary method for automatically segmenting an object of interest in accordance with various embodiments.

FIG. 2 is another flowchart of a portion of the method shown in FIG. 1 in accordance with various embodiments.

FIG. 3 is an exemplary coronal image that may be generated in accordance with various embodiments.

FIG. 4 is an exemplary axial image that may be generated in accordance with various embodiments.

FIG. 5 is another exemplary coronal image that may be generated in accordance with various embodiments.

FIG. 6 is another exemplary axial image that may be generated in accordance with various embodiments.

FIG. 7 is still another exemplary coronal image that may be generated in accordance with various embodiments.

FIG. 8 is another exemplary axial image that may be generated in accordance with various embodiments.

FIG. 9 is still another exemplary coronal image that may be generated in accordance with various embodiments.

FIG. 10 is still another exemplary coronal image that may be generated in accordance with various embodiments.

FIG. 11 is another flowchart of a portion of the method shown in FIG. 1 in accordance with various embodiments.

FIG. 12 is a pictorial view of an exemplary imaging system formed in accordance with various embodiments.

### DETAILED DESCRIPTION OF THE INVENTION

The foregoing summary, as well as the following detailed description of various embodiments, will be better understood when read in conjunction with the appended drawings. To the extent that the figures illustrate diagrams of the functional blocks of the various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

Described herein are various embodiments for automatically detecting an organ of interest that may be applied to, or used with, information acquired from a plurality of imaging modalities. The imaging modalities include, for example, a positron emission tomography (PET) system, a single photon emission computed tomography (SPECT) system, a computed tomography (CT) system, an ultrasound system, an x-ray system, a magnetic resonance (MR) system, and/or other imaging systems. In operation, the various embodiments, automatically detect and provide a visual indication of the organ of interest using for example, a region of interest (ROI) that is placed in the organ of interest. As a result, the ROI facilitates identifying voxels that belong to the organ of interest. Accordingly, in various embodiments, the organ of interest is automatically detected or identified by automatically adapting the parameters to be utilized with different modalities even for functional images such as PET modality.

In various embodiments, the methods and systems described herein automatically provide the user with a location of the organ of interest. The user may either accept or reject the automatically determined location based on inputs entered by the user. At least one technical effect of some embodiments includes improving the identification of the organ of interest. The improved identification of the organ of interest may then be utilized to, for example, improve an accuracy of the segmentation process and/or reduce a time to perform the organ detection. For example, in various embodiments, the methods described herein for automatic organ detection may be performed on a system while the system is performing other imaging tasks. The results of the organ detection may then be displayed to a user when the segmentation is completed or when desired to be viewed by the user. Additionally, automatic liver detection, for example when used in a PET Oncology procedure, provides a reference region for normalizing Fluorodeoxyglucose (FDG) uptake from a baseline to a follow up comparison of a standard uptake value (SUV). The methods described herein also facilitate reducing a time to perform radiation therapy planning, surgical planning, therapy monitoring, etc.

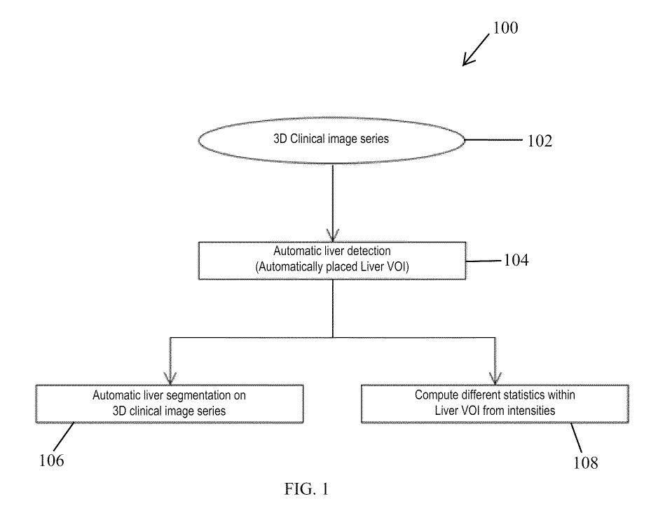

FIG. 1 is a flowchart of an exemplary method 100 for automatically identifying an object of interest and displaying the object of interest. In the various embodiments, the method 100 is embodied as an algorithm. The method 100 and/or the algorithm may be embodied as a set of instructions that are stored on a computer and implemented using, for example, a module 550, shown in FIG. 12, which may be software, hardware, a combination thereof, or a tangible non-transitory computer readable medium.

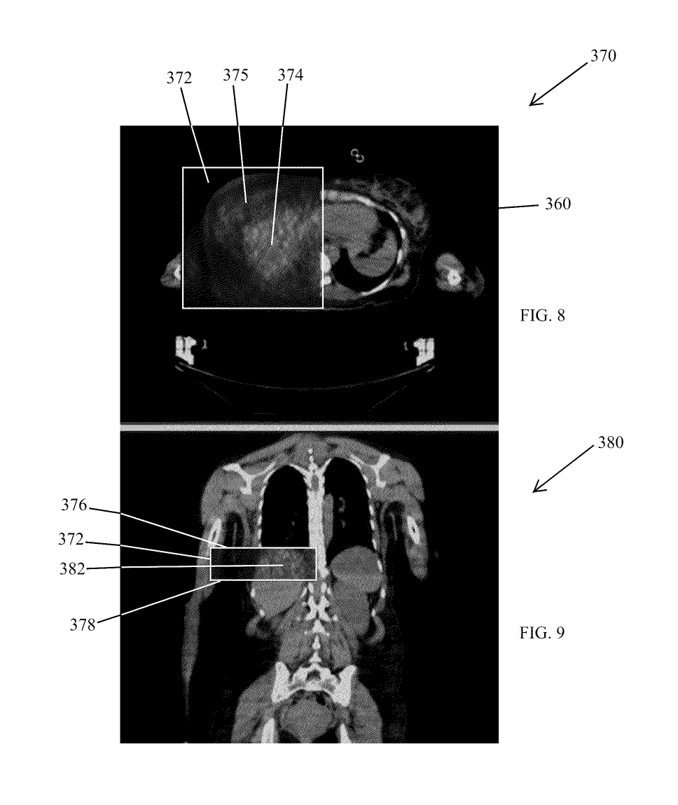

Referring again to FIG. 1, the method 100 includes obtaining at 102, a three-dimensional (3D) image dataset, such as the image dataset 504 shown in FIG. 12, of an object of interest, such as the patient 506 shown in FIG. 12. The image dataset 504 may be acquired by retrieving the image dataset 504 from a database or, alternatively, receiving the image dataset 504 from an imaging system. The image dataset 504 may include, for example, a series of medical images taken along an examination axis. In various embodiments, the series of medical images may include a series of cross-sectional images of an organ of interest, such as for example, the patient's liver 375 (shown in FIG. 8). Although various embodiments are described herein for utilizing PET data to detect the patient's liver, it should be realized that the methods described herein may be utilized with image data acquired from a plurality of medical imaging modalities, and a PET imaging system is one such modality.

At 104, the liver is automatically detected. In various embodiments, the automatic liver detection is performed using the automatically accessed image dataset 504. More specifically, the method 100 enables fully automatic detection of the organ of interest, for example, the liver. Accordingly, while various embodiments are described with respect to automatically detecting a liver, it should be realized that other objects and/or organs of interest may be detected. For example, such objects or organs may include metastatic lesions in the bone or the brain. If the liver is diseased, a reference region in the blood pool from the descending aorta may be detected. The organ to be detected may be based on the specific tracer being utilized during the examination.

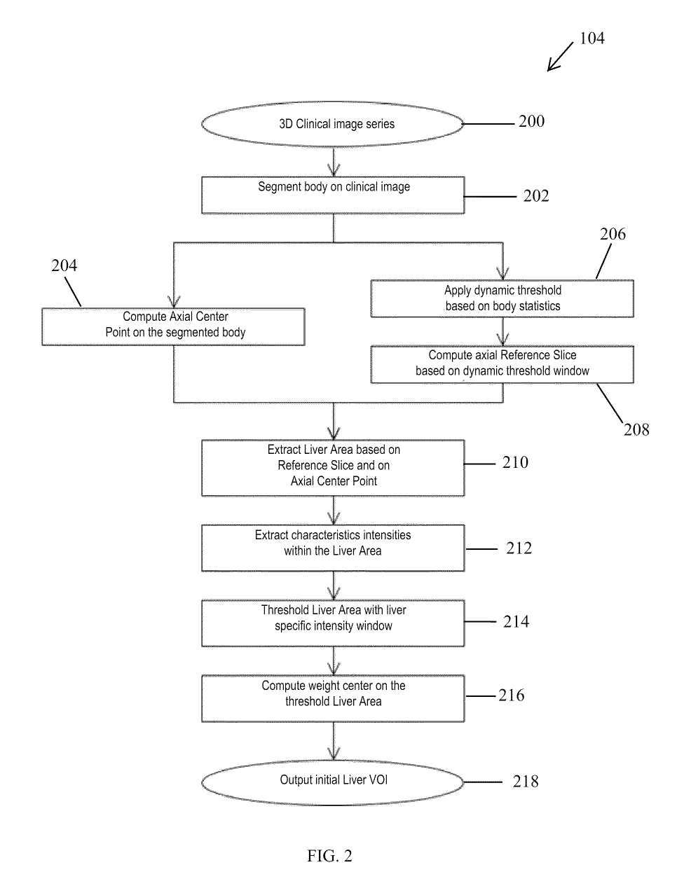

FIG. 2 is a flowchart illustrating an exemplary method of implementing step 104 shown in FIG. 1. At 200 the image dataset 504 acquired at 102 is input to, or obtained by, the module 550. As described above, the image dataset 504 may be either anatomical images acquired from, for example a CT imaging system or an MR imaging system. The image dataset 504 may also be functional images acquired from, for example, a PET imaging system or a SPECT imaging system. In the various embodiments described herein, the image dataset 504 is acquired with the patient laying in the supine position such that the liver is aligned on the left side of the body (HFS). Optionally, the image dataset 504 may be acquired with the patient laying in the prone position. Accordingly, if the patient is in the prone position, the image dataset 504 may be transformed, i.e. by inverting the images, such that the liver is on the left side of the images.

At 202, a body segmentation is performed. Segmentation is used to outline objects and/or regions within the image dataset 504. In various embodiments described herein, the segmentation is utilized to identify an outline of the body of the patient being imaged. For example, FIG. 3 is an exemplary image 300 of a body 302 that may be segmented using the image data 504 input at 200. The segmentation may be performed using a segmentation algorithm stored, for example, on the module 550. The segmentation algorithm uses a principle, whereby it is generally assumed that various organs, tissue, fluid, and other anatomical features may be differentiated by determining the density of each voxel in the image dataset 504. The density generally represents the intensity value of the voxel. Based on the density values of each of the voxels, the patient's body 302 may be distinguished from non body voxels (background). Accordingly, at 202 the segmentation algorithm is configured to automatically compare the density value for each voxel in the image dataset 504 to a predetermined density value, using for example, a thresholding process. In one exemplary embodiment, the predetermined density value may be a range of predetermined density values. The predetermined density value range may be automatically set based on a priori information of the patient's body 302. Optionally, the predetermined range may be manually input by the operator. In one embodiment, if the density value of a voxel is within the predetermined range, the voxel is classified as belonging to the patient's body 302. Otherwise, the voxel is classified as a background voxel that does not form part of the patient's body 302 as shown in FIG. 3 as dark or black pixels. It should be realized that the segmentation algorithm may also be utilized with other segmentation techniques to identify the patient's body 302. Additionally, as should be appreciated, other suitable segmentation algorithms may be used. Accordingly, at 202, voxels that define the patient's body 302 are segmented with respect to voxels that are not part of the patient's body.

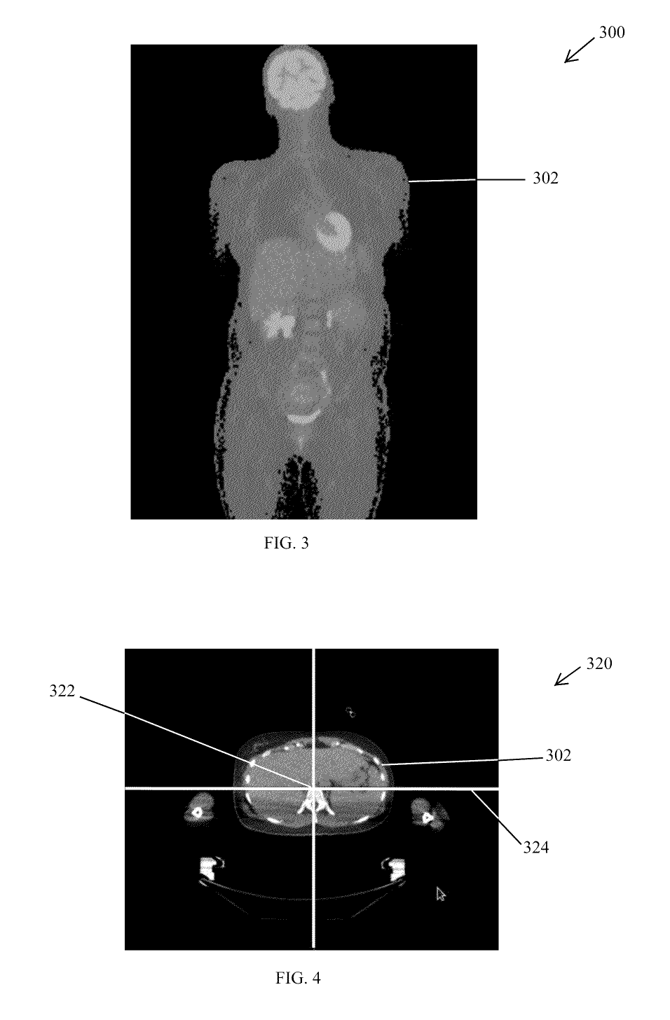

At 204, an axial center point of the patient's body 302 is calculated. The axial center point represents a center of mass of the patient's body 302. For example, FIG. 4 is an exemplary axial image 320 of the body 302 that may be segmented using the image data 504 input at 200. As shown in FIG. 4, the image 320 includes an exemplary center point 322, illustrated using a pair of cross-hairs 324, that indicate an axial center of mass of the patient's body 302 as calculated at 204. In various embodiments, the axial center point 322 is calculated using the voxels that are defined as part of the patient's body at 202 and shown in FIG. 3.

In operation, the axial center point 322 may be calculated using the voxels that belong to the body 302. More specifically, the center point 322 may be calculated by identifying the edges or boundaries of the body 302. The edges or boundaries may then be utilized to determine various axial distances in an x-direction and a y-direction along the body 302. The axial distances may then be utilized to calculate the axial center point 322 as a single x,y value that represents the center of mass of the body 302. In various embodiments, the image dataset 504 is a 3D image dataset. Accordingly, a center point 322 may be calculated for each image in the image dataset 504.

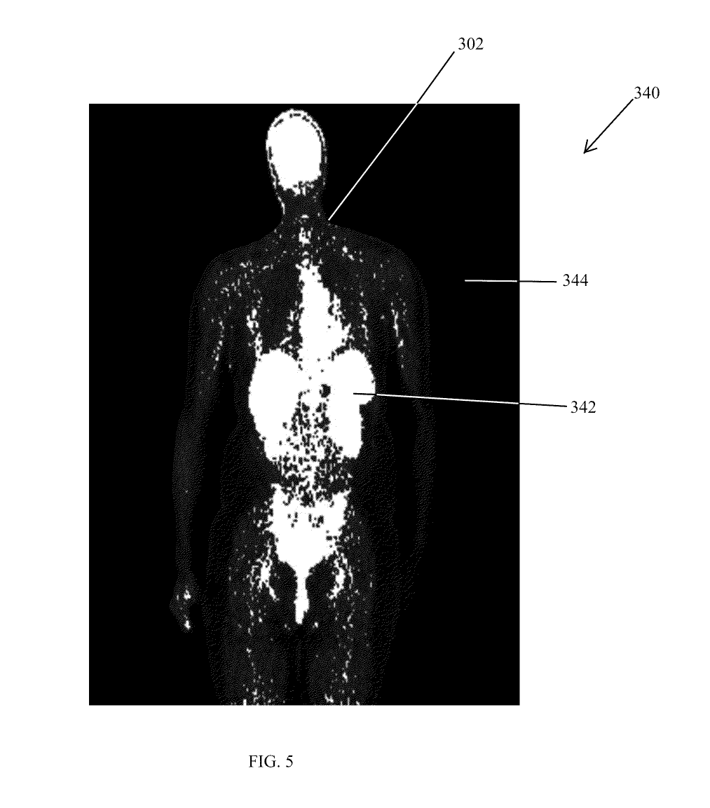

Referring again to FIG. 2, at 206 a dynamic threshold is applied to the segmented body 302 acquired at 202. More specifically, the dynamic threshold facilitates differentiating voxels that represent different soft tissues within the body 302. For example, FIG. 5 illustrates an exemplary image 340 of the body 302 that illustrates the differentiation between various soft tissues 342 (shown in white) and a background region 344 (shown in black) of the body 302. In various embodiments, the dynamic threshold may be calculated utilizing a histogram (not shown) of the body 302. For example, a histogram of the body 302 is generated using the image dataset 504. The histogram may then be utilized to calculate a threshold that differentiates the body 302 from the voxels forming the background region of the image as shown in FIG. 3. More specifically, the histogram may be utilized to identify the contours or outline of the body 302. In various embodiments, the dynamic threshold is a single value, derived using the information on the histogram, that represents a differentiation between the soft tissue 342 and the background region 344 of the image 340.

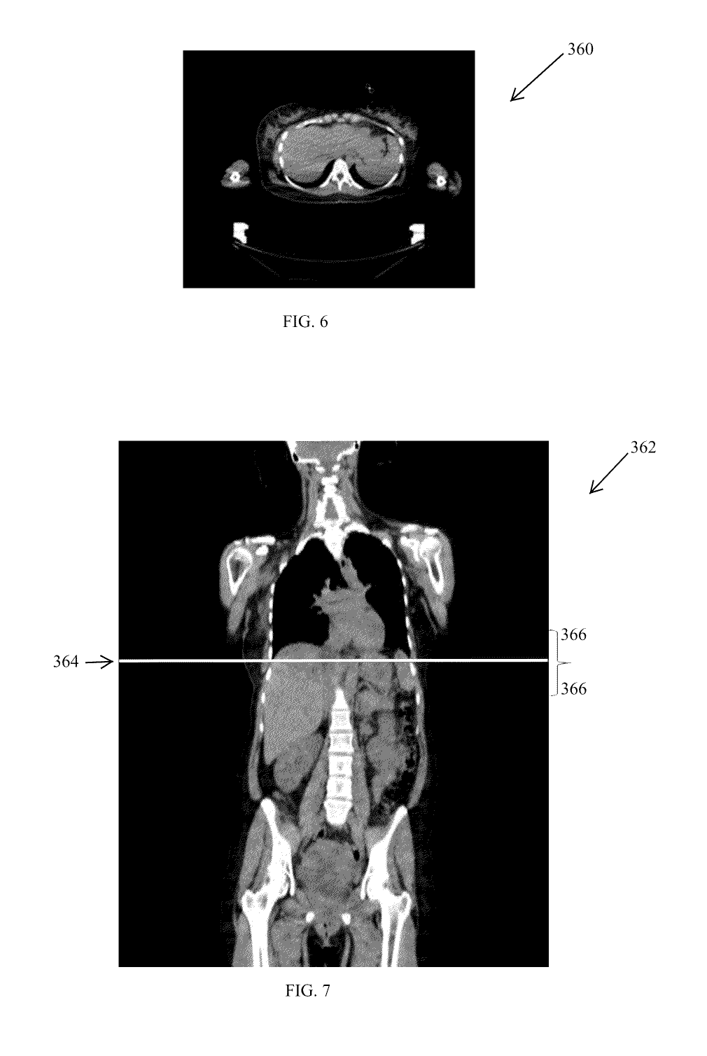

Referring again to FIG. 2, at 208 the dynamic threshold calculated at 204 is utilized to select an axial reference slice, such as an axial reference slice 360 shown in FIG. 6. FIG. 7 is a coronal image 362 that illustrates a position of the axial reference slice 360 within the image dataset 504. It should be noted that the line 364 represents the axial location of the reference slice 360 within the image dataset 504. The soft tissue facilitates locating the abdomen area or more precisely a specific range of the liver.

In various embodiments, the liver may be automatically identified based on a priori information of the liver. For example, the module 550 may utilize a priori information of the liver to identify the liver within the image dataset 504. Such a priori information may include, for example, an expected liver intensity. The a priori information may also include information of various liver studies that have been previously performed. Based on the previous studies, the a priori information may include pixel intensity values that represent known livers. Thus, the module 550 may have information of pixel intensity values that more than likely represent pixels of the liver, the module 550 may utilize this information to locate the liver. In various embodiments, each image in the image dataset 504 is thresholded, using the a priori pixel intensity values of a liver, to identify whether that particular slice includes a portion of the liver.

The module 550 may also be configured to automatically access a predetermined range of pixel densities that are associated with the liver. The module 550 then searches the image dataset 504, on a slice-by-slice basis, to identify all pixels having an intensity that falls within the predetermined range. In other embodiments, the a priori information may include soft tissue intensity values of areas known to surround the liver as identified at 206. The reference slice 360 may then be selected based on the intensity values thresholded at 206.

Accordingly, at 208 the module 550 automatically selects a single slice or image that best represents the liver. For example, as described above, FIG. 6 illustrates an exemplary reference slice 360 that best represents the liver. In various embodiments, the term “best represents” as used herein means the slice that includes a representation of the liver that meets the largest number of criteria from a list of criteria. The list of criteria may include, for example, the slice that shows the largest area of the liver or the best view of the liver. It should be realized that the a priori information may also include the type of examination being performed. As a result, the system receives inputs identifying the types of images that the operator may need to perform a specific type of diagnosis. Accordingly, the module 550 is configured to automatically access a priori information on the type, view, etc., of the liver that the operator is requesting. The module 550 may utilize this a priori information to identify a single slice, e.g. the reference slice 360 that shows the liver from a view that best enables the operator to perform the diagnosis. In various embodiments, step 204 may be performed concurrently with steps 206 and 208. Optionally, step 204 may be performed before or after steps 206 and 208.

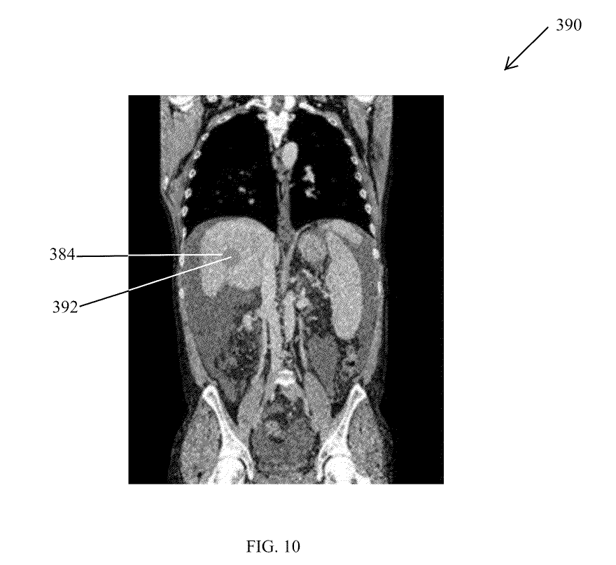

Referring again to FIG. 2, at 210 the axial center point 322 determined at 204 and the reference slice 360 selected at 208 are utilized to determine a location of the liver and also to determine the physical boundaries or extent of the liver. More specifically, as described above, the center point 322 represents the center of mass of the body 302. The center point 322 also enables the module 550 to differentiate the left and right sides of the body 302. For example, it is known that the liver is on the left side of the body 302. Accordingly, in various embodiments to extract a liver region of interest (ROI) 372 (shown in FIGS. 8 and 9), a predetermined quantity of slices is selected above and below the reference slice 360, in an axial direction. For example, as shown in FIG. 7, the location of the reference slice 360 is previously determined at 206. Accordingly, a predetermined number of slices 366, as shown in FIG. 7, may be selected on both sides of the reference slice 360. The reference slice 360 and the predetermined slices 366 are then utilized to calculate the liver ROI 372. For example, assume that the reference slice 360 is the 100^{th} slice in the image dataset 504. Slices 80-99 and 101-140 may therefore be selected as the slices 366.

In various embodiments, the output from step 210 is the liver (ROI) 372 having at least a portion of the liver therein. For example, FIG. 8 is an axial image 370 that illustrates the reference slice 360 including the liver ROI 372 that is drawn in 2D around a plurality of liver voxels 374 of a liver 375. The liver ROI 372 may be displayed to user via an overlay (as shown with green overlay in FIG. 8) or any other visual indicator. Moreover, FIG. 9 is a coronal image 380 that illustrates the liver ROI 372. As shown in FIG. 9, the liver ROI 372 includes an upper boundary 376 that represents the last slice, i.e. the 80^{th} slice, in the set of predetermined slices 366 above the reference slice 360 and a lower boundary 378 that represents the last slice, i.e. the 140^{th} slice, in the set of predetermined slices 366 below the reference slice 360. It should be realized that although a 2D liver ROI 372 is illustrated, that in the exemplary embodiment, the liver ROI 372 is a 3D boundary.

Referring again to FIG. 2, at 212 characteristic intensities of the liver 375 are extracted using the information within the liver ROI 372 described above. In various embodiments, an intensity based analysis of voxels within the liver ROI 372 is utilized to identify the liver voxels 374 that form part of the liver 375 and which voxels form part of the background, or non-liver portions, surrounding the liver 375. In the exemplary embodiment, a liver voxel 374 is identified using a priori knowledge. For example, the voxels within the liver ROI 372 may be compared to an intensity value of a known liver voxel. Voxels that are within a predetermined range of the known liver voxel intensity value may be classified as liver voxels 374 and voxels that are outside the predetermined range may be classified as non-liver voxels. However, in other embodiments, the identification of the liver voxel 374 is not based on a priori knowledge, rather other methods may be utilized.

Referring again to FIG. 2, at 214 an initial segmentation of the liver ROI 372 is performed based on the liver voxels 374 identified at 212. Thus, at 214 the liver voxels 374 within the liver ROI 372 are separated or segmented from the voxels defined as not liver based in the intensities extracted at 212.

At 216, a center of mass 382 of the liver ROI 372 is calculated. As described above, the voxels 374 representing the liver 375 are identified. Accordingly, at 216 the voxels 374 defined as the liver 375 are utilized to determine the center of mass 382 of the liver 375. In various embodiments, the center of mass 382 of the liver 375 may be calculated using the same method of finding the axial center point 322 as described above at step 204. However, it should be realized that at 216, the center of mass 382 is calculated in a 3D coordinate system, (x,y,z). For example, the center of mass 382 of the liver 375 may be calculated by determining the edges or boundaries of the liver 375. The edges or boundaries of the liver 375 may then be utilized to determine various axial distances in an x-direction, a y-direction, and a z-direction along the liver 375. The axial distances may then be utilized to calculate an axial center point or the center of mass 382 (shown in FIG. 9) as a single x,y,z value that represents the center of mass 382 of the liver 375. In various embodiments, the image dataset 504 is a 3D image dataset. Accordingly, the center of mass point 382 may be calculated for each image in the image dataset 504.

Referring again to FIG. 2, a liver volume of interest (VOI) 384 is output. For example, FIG. 10 is coronal image 390 of the liver volume of interest 384 that may be generated as described above. In various embodiments, the liver VOI 384 represents the segmented liver 375 and information representing the center of mass 382 of the liver 375. In various embodiments, the liver VOI 384 may be a small sphere, as shown in FIG. 10, having a 3D center point 392 which represents the liver VOI 384 location. Accordingly, in use the methods described herein may be utilized to further modify this location inside the liver 375, i.e. to place/relocate the liver VOI 384 to the optimal location inside the liver 375 to avoid lesions and organ boundary voxels.

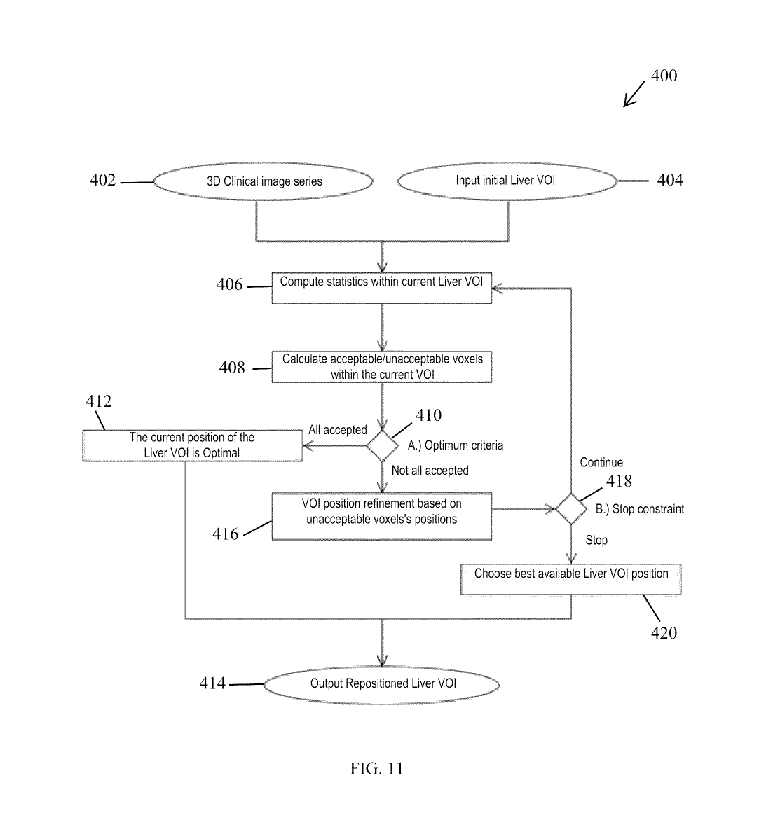

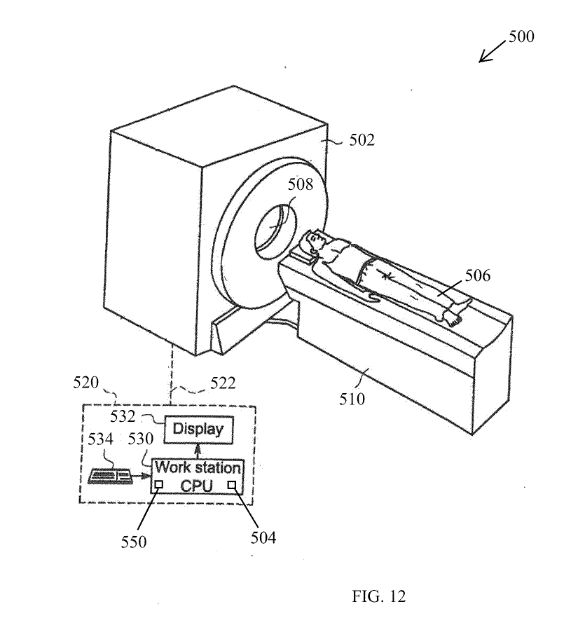

For example, FIG. 11 is a flowchart of a method 400 for refining or adjusting a location of the liver VOI 384 (shown in FIG. 10), i.e. the segmented liver volume, by for example, detecting lesions and organ edges within the liver VOI 384. More specifically, the method 400 facilitates adjusting the liver VOI 384 such that the liver 375 is more centered within the a subsequent liver VOI and such that the liver 375 is substantially defined within the subsequent liver VOI as described in more detail below.

At 402, the image dataset 504 is input to the module 550. At 404, the liver VOI 384, including the center of mass 382, identified using the method 200 is input to the module 550. At 406, statistics are calculated using the voxels within the liver VOI 384. In various embodiments, the statistics may include for example, an average of the voxel intensity values within the liver VOI 384, a mean value of the voxel intensity values within the liver VOI 384, and/or a variation of the voxel intensity values within the liver VOI 384.

At 408, each of the voxels in the liver VOI 384 is classified as either an acceptable voxel or an unacceptable voxel. As used herein, an acceptable voxel is a voxel that represents healthy liver tissue and an unacceptable voxel represents a diseased tissue, a tumorous tissue, and/or a non-liver tissue. In various embodiments, the acceptable and non-acceptable voxels are determined based on an intensity of the voxels within the liver VOI 384. For example, in various embodiments, if the voxel is within a predetermined range of the average, mean, or variance of the statistics calculated at 406, the voxel is classified as an acceptable voxel meaning that more likely than not, the voxel is part of a healthy liver. Optionally, if the voxel intensity is outside the predetermined range of the average, mean, or variance of the statistics calculated at 406, the voxel is classified as an unacceptable voxel meaning that more likely than not, the voxel is not part of a healthy liver.

At 410, a predetermined criteria is applied to the voxels classified at 408. In operation, the predetermined criteria defines whether the quantity of acceptable voxels is greater than a predetermined threshold and determines if a location of the liver VOI 384 is optimal, that is the liver VOI 375 is substantially within the liver 375.

For example, assume that 98% percent of the voxels are classified as acceptable voxels, i.e. 2% are unacceptable voxels at 408. Moreover, assume that the predetermined threshold is 95% meaning that liver VOI 384 is at an optimal position. Accordingly, the quantity of acceptable voxels, in this example, is greater than the predetermined quantity of voxels indicating that the liver 375 is at the optimal position.

In one embodiment, if the liver VOI 384 is at the optimal position, i.e. more than 95% of the voxels are acceptable, the method proceeds step 412 wherein the method 400 determines that no further adjustment of the liver VOI 384 is required because the liver VOI 384 is currently in the optimal position. At 414, the method is terminated and the liver VOI 384 may be utilized for further processing, such as for example, to segment the liver or to reconstruct an image of the liver 375 as shown in step 106 or to compute statistics of the liver as shown in step 108, both shown in FIG. 1.

In another embodiment, if the liver VOI 384 is not at the optimal position, i.e. less than 95% of the voxels are acceptable, the method proceeds to step 416 wherein a position of the liver VOI 384 is adjusted. More specifically, at 416 the liver VOI 384 is moved to a second position. For example, the liver VOI 384 may be moved in a first direction from the initial position of the liver VOI 384 shown in FIG. 10. At 418, a stop constraint is analyzed to determine if the method 400 has exceeded a predetermined quantity of iterations or exceeded a predetermined time threshold. The stop constraint is explained in more detail below. In the exemplary embodiment, assume that the stop constraint has not been exceeded. Accordingly, as shown in FIG. 11 the method 400 performs a second iteration of steps 404-410. Accordingly, at 404 the liver VOI 384 at the revised or second position is input. At 406 the statistics are calculated for the liver VOI 384 at the second position. At 408, acceptable and unacceptable voxels are determined at the second position. At 410, the optimal criteria are then applied to the voxels within the liver VOI 384 at the second position. If the voxels exceed the predetermined criteria, the method proceeds to steps 412 and 414 as described above.

Optionally, the method proceeds again to step 416. For example, assume that 98% percent of the voxels are classified as acceptable voxels, i.e. 2% are unacceptable voxels at 408. Moreover, assume that the predetermined threshold is 95% meaning that the liver VOI 384 is at an optimal position. Accordingly, the quantity of acceptable voxels at the second position is greater than the predetermined quantity of voxels indicating that the liver 375 is at the optimal position.

In one embodiment, if the liver VOI 384 is at the optimal position, i.e. more than 95% of the voxels are acceptable, the method proceeds step 412 wherein the method 400 determines that no further adjustment of the liver VOI 384 is required because the liver VOI 384 is in the optimal position. At 414, the method is terminated and the liver VOI 384 may be utilized for further processing, such as for example, to reconstruct an image of the liver 375.

However, if the voxels do not exceed the predetermined criteria, the quantity of acceptable voxels at the second position is compared to the quantity of voxels at the initial position. For example, as described above, assume that the quantity of acceptable voxels at the initial position is 90%. Moreover, assume that the quantity of acceptable voxels at the second or revised position is 85%. Thus, by moving the liver VOI 384 is from the initial position, in the first direction to the second position, the quantity of acceptable voxels has decreased. Accordingly, at 416, the liver VOI 384 is moved in a second direction, that is different to the first direction to a third position. A third iteration of steps 404-416 is then performed as described above. In various embodiments, the method is configured to perform multiple iterations until either the quantity of acceptable voxels exceeds the predetermined threshold or the stop constraint at 418 is exceeded.

In operation, the method described herein generally investigates the voxels in the current liver VOI 384. If there are voxels that do not behave as healthy tissue, then the current liver VOI 384 is not in an optimal position. The method further investigates the position of these not acceptable voxels and also investigates the vicinity of the current liver VOI 384 and then calculates one or more possible next locations based on this information. In the exemplary embodiment, if a better position is identified, the liver VOI 384 is relocated to the better position. If the next position is worse than the current position, the liver VOI 384 may be moved to next position to improve the results later. It should be realized that the information obtained at each location is stored to enable the method to identify the best location. Therefore, the final results of the method are the current position despite the decision to move out from that position. When the predetermined quantity of iterations is completed, or the predetermined time limit is exceeded, and no optimal location is identified, the best location from the previously visited locations is selected.

In various embodiments, the stop constraint at 418 may be based on a quantity of iterations. For example, in various embodiments, steps 404-416 may be performed for a predetermined quantity of iterations. In one embodiment, if the quantity of acceptable voxels does not exceed the predetermined threshold in any of the iterations, then at 420, the method is configured to select the liver volume having the largest quantity of acceptable voxels. For example, assume that the stop constraint is set to five iterations. Moreover, assume that the first iteration indicates that 90% of the voxels are acceptable voxels, the second iteration indicates that 85% of the voxels are acceptable voxels, the third iteration indicates that 91% of the voxels are acceptable voxels, the fourth iteration indicates that 92% of the voxels are acceptable voxels, and the fifth iteration indicates that 94% of the voxels are acceptable voxels. Accordingly, in the exemplary embodiment, a volume of interest inside the liver is acquired during the fifth iteration, wherein 94% of the voxels are acceptable voxels is used at 414.

In various other embodiments, the stop constraint at 418 is a time constraint. For example, assume that the stop constraint is set to one second. Moreover, assume, as discussed above, that five iterations are performed during the one second time period wherein first iteration indicates that 90% of the voxels are acceptable voxels, the second iteration indicates that 85% of the voxels are acceptable voxels, the third iteration indicates that 91% of the voxels are acceptable voxels, the fourth iteration indicates that 92% of the voxels are acceptable voxels, and the fifth iteration indicates that 94% of the voxels are acceptable voxels. Accordingly, in the exemplary embodiment, each of the volumes acquired during the one second period, or prior to the expiration of the time constraint is analyzed to determine which iteration generated a liver volume having the greatest quantity of acceptable voxels. In the exemplary embodiment, the liver volume acquired during the fifth iteration, wherein 94% of the voxels are acceptable voxels is used at 414. It should be realized that in various other embodiments, the stop constraint at 418 may be a manual stop constraint. More specifically, the user may manually stop the module 550 at any time during the iterations. In this example, the module 550 is then configured to select the iteration having the greatest quantity of acceptable voxels as described above. Optionally, the user may manually select the volume.

A technical effect of various embodiments described herein is to provide a fully automatic detection algorithm. The detection algorithm is configured to operate in real-time. Moreover, the detection algorithm may be utilized with a variety of image datasets acquired from a plurality of imaging modalities. Moreover, the detection algorithm may be utilized with contrast enhanced and non-contrast enhanced images.

Various embodiments described herein provide an imaging system 500 as shown in FIG. 12. In the illustrated embodiment, the imaging system 500 is a stand-alone PET imaging system. Optionally, the imaging system 500 may be embodied, for example, as a CT imaging system, an MRI system, or a SPECT system. The various embodiments described herein are not limited to standalone imaging systems. Rather, in various embodiments, the imaging system 500 may form part of a multi-modality imaging system that includes the PET imaging system 500 and a CT imaging system, an MRI system, or a SPECT system, for example. Moreover, the various embodiments are not limited to medical imaging systems for imaging human subjects, but may include veterinary or non-medical systems for imaging non-human objects, etc.

The imaging system 500 includes a gantry 502. The gantry 502 is configured to acquire the image dataset 504. During operation, a patient 506 is positioned within a central opening 508 defined through the gantry 502, using, for example, a motorized table 510. The imaging system 500 also includes an operator workstation 520. During operation, the motorized table 510 moves the patient 506 into the central opening 508 of the gantry 502 in response to one or more commands received from the operator workstation 520. The workstation 520 then operates both the gantry 502 and the table 510 to both scan the patient 506 and acquire the image dataset 504 of the patient 506. The workstation 520 may be embodied as a personal computer (PC) that is positioned near the imaging system 500 and hard-wired to the imaging system 500 via a communication link 522. The workstation 520 may also be embodied as a portable computer such as a laptop computer or a hand-held computer that transmits information to, and receives information from, the imaging system 500. Optionally, the communication link 522 may be a wireless communication link that enables information to be transmitted to or from the workstation 520 to the imaging system 500 wirelessly. In operation, the workstation 520 is configured to control the operation of the imaging system 500 in real-time. The workstation 520 is also programmed to perform medical image diagnostic acquisition and reconstruction processes described herein.

In the illustrated embodiment, the operator workstation 520 includes a central processing unit (CPU) or computer 530, a display 532, and an input device 534. As used herein, the term “computer” may include any processor-based or microprocessor-based system including systems using microcontrollers, reduced instruction set computers (RISC), application specific integrated circuits (ASICs), field programmable gate array (FPGAs), logic circuits, and any other circuit or processor capable of executing the functions described herein. The above examples are exemplary only, and are thus not intended to limit in any way the definition and/or meaning of the term “computer”. In the exemplary embodiment, the computer 530 executes a set of instructions that are stored in one or more storage elements or memories, in order to process information, such as the image dataset 504. The storage elements may also store data or other information as desired or needed. The storage element may be in the form of an information source or a physical memory element located within the computer 530.

In operation, the computer 530 connects to the communication link 522 and receives inputs, e.g., user commands, from the input device 534. The input device 534 may be, for example, a keyboard, mouse, a touch-screen panel, and/or a voice recognition system, etc. Through the input device 534 and associated control panel switches, the operator can control the operation of the PET imaging system 500 and the positioning of the patient 506 for a scan. Similarly, the operator can control the display of the resulting image on the display 532 and can perform image-enhancement functions using programs executed by the computer 530.

The imaging system 500 also includes a segmentation module 550 that is configured to implement various methods, such as the methods 200 and 400, as described herein. The segmentation module 550 may be implemented as a piece of hardware that is installed in the computer 530. Optionally, the module 550 may be implemented as a set of instructions that are installed on the computer 530. The set of instructions may be stand-alone programs, may be incorporated as subroutines in an operating system installed on the computer 530, may be functions in an installed software package on the computer 530, and the like. It should be understood that the various embodiments are not limited to the arrangements and instrumentality shown in the drawings.

The set of instructions may include various commands that instruct the module 550 and/or the computer 530 as a processing machine to perform specific operations such as the methods and processes of the various embodiments described herein. The set of instructions may be in the form of a non-transitory computer readable medium. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the phrase “reconstructing an image” is not intended to exclude embodiments of the present invention in which data representing an image is generated, but a viewable image is not. Therefore, as used herein the term “image” broadly refers to both viewable images and data representing a viewable image. However, many embodiments generate, or are configured to generate, at least one viewable image.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for automatically detecting or displaying an organ of interest, said method comprising:
accessing a medical image dataset using a processor;
automatically segmenting the medical image dataset to identify an outline of a body of a patient;
automatically determining an axial reference image slice and an axial center point using the segmented body of the patient;
automatically determining a location of the organ of interest using the axial reference image slice and the axial center point; and
automatically placing a visual indicator in the organ of interest based on the determined location.

2. The method of claim 1, wherein the axial center point is located at a center of mass of the body of the patient.

3. The method of claim 1, wherein the organ of interest is a liver.

4. The method of claim 1, further comprising:
generating a liver volume of interest (VOI) using the axial reference image slice;
performing an intensity based analysis of voxels within the liver VOI; and
classifying voxels within the liver VOI as either liver voxels or non-liver voxels based on the intensity based analysis.

5. The method of claim 4, further comprising calculating a center of mass of a liver using the voxels classified as liver voxels.

6. The method of claim 1, further comprising:
calculating at least one of a mean voxel value, an average voxel value, and a variation value using the voxels within the a liver volume of interest (VOI); and
automatically adjusting a position of the liver VOI based on the calculated mean, average, or variation values.

7. The method of claim 1, further comprising:
generating a liver volume of interest (VOI) using the axial reference image slice;
identifying a quantity of acceptable liver voxels within the liver VOI;
comparing the identified quantity of acceptable liver voxels to a predetermined threshold; and
repositioning the liver VOI to a second different position based on the comparison.

8. The method of claim 1, further comprising:
generating a liver volume of interest (VOI) using the axial reference image slice; and
iteratively moving the liver VOI to a different position until a quantity of acceptable liver voxels within the liver VOI exceeds a predetermined threshold.

9. The method of claim 1, further comprising:
generating a liver volume of interest (VOI) using the axial reference image slice; and
iteratively repositioning the liver VOI to a different position until a quantity of acceptable liver voxels exceeds a predetermined threshold or until a time constraint is exceeded.

10. A medical imaging system comprising:
a detector array; and
a computer coupled to the detector array, the computer configured to
access a medical image dataset using a processor;
automatically segment the medical image dataset to identify an outline of a body of a patient;
automatically determine an axial reference image slice and a axial center point using the segmented body of the patient; and
automatically determine a location of a liver using the axial reference image slice and the axial center point.

11. The medical imaging system of claim 10, wherein the axial center point is located at a center of mass of the body of the patient.

12. The medical imaging system of claim 10, wherein the computer is further configured to:
automatically generate a liver volume of interest (VOI) using the axial reference image slice;
automatically perform an intensity based analysis of voxels within the liver VOI; and
automatically classify voxels within the liver VOI as either liver voxels or non-liver voxels based on the intensity based analysis.

13. The medical imaging system of claim 10, wherein the computer is further configured to:
calculate at least one of a mean voxel value, an average voxel value, and a variation value using the voxels within the liver VOI; and
automatically adjust a position of the liver VOI based on the calculated mean, average, or variation values.

14. The medical imaging system of claim 10, wherein the computer is further configured to:
generate a liver volume of interest (VOI) using the axial reference image slice;
identify a quantity of acceptable liver voxels within the liver VOI;
compare the identified quantity of acceptable liver voxels to a predetermined threshold; and
reposition the liver VOI to a second different position based on the comparison.

15. The medical imaging system of claim 10, wherein the computer is further configured to:
generate a liver volume of interest (VOI) using the axial reference image slice; and
iteratively move the liver VOI to a different position until a quantity of acceptable liver voxels within the liver VOI exceeds a predetermined threshold.

16. The medical imaging system of claim 10, wherein the computer is further configured to
generate a liver volume of interest (VOI) using the axial reference image slice; and
iteratively reposition the liver VOI to a different position until a quantity of acceptable liver voxels exceeds a predetermined threshold or until a time constraint is exceeded.

17. A non-transitory computer readable medium being programmed to instruct a computer to:
access a medical image dataset using a processor;
automatically segment the medical image dataset to identify an outline of a body of a patient;
automatically determine an axial reference image slice and a axial center point using the segmented body of the patient, wherein the axial center point is located at a center of mass of the body of the patient; and
automatically determine a location of a liver using the axial reference image slice and the axial center point.

18. The non-transitory computer readable medium of claim 17, being further programmed to:
automatically generate a liver volume of interest (VOI) using the axial reference image slice;
automatically perform an intensity based analysis of voxels within the liver VOI; and
automatically classify voxels within the liver VOI as either liver voxels or non-liver voxels based on the intensity based analysis.

19. The non-transitory computer readable medium of claim 17, being further programmed to:
calculate at least one of a mean voxel value, an average voxel value, and a variation value using the voxels within the liver VOI; and
automatically adjust a position of the liver VOI based on the calculated mean, average, or variation values.

20. The non-transitory computer readable medium of claim 17, being further programmed to:
generate a liver volume of interest (VOI) using the axial reference image slice; and
iteratively reposition the liver VOI to a different position until a quantity of acceptable liver voxels exceeds a predetermined threshold or until a time constraint is exceeded.
